(19) United States
(12) Patent Application Publication
(10) Pub. No.: US 2005/0267421 A1
(43) Pub. Date: Dec. 1, 2005

(54) Catheter cleaner

(76) Inventor: Thomas W. Wing, Claremont, CA (US)

Correspondence Address: Kit M. Stetina, Esq.;STETINA BRUNDA GARRED & BRUCKER, Suite 250, 75 Enterprise, Aliso Viejo, CA, 92656, US

(21) Appl. No.: 10/856,031

(22) Filed: May 28, 2004

(51) Int. Cl. A61M 25/00

(52) U.S. Cl. 604/267

(57) ABSTRACT

A catheter cleaner comprising a head, rod, and a handle is provided. The head, rod and handle may be fixedly attached to each other such that manipulation of the handle may also control movement of the head, both in the linear direction as well as a rotational movement. The head may define a tip and cutting edges. The tip may have a curved configuration which may be operative to assist in the insertion of the cleaner into and indwelling catheter and, the cutting edges may be operative to dislodge debris from an inside wall of the indwelling catheter. Moreover, the tip may be used to push against debris lodged or attached to the inside wall of the indwelling catheter to dislodge the same.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND OF THE INVENTION

The present invention relates generally to a catheter cleaner, and more particularly to a cleaning device capable of removing debris from an inner diameter of an indwelling catheter.

An indwelling catheter is a slender, flexible tube inserted into a bodily channel, such as a G-tube or urethra, to distend or maintain an opening to an internal cavity. For example, the indwelling catheter may be used to assist patients who are having difficulty urinating. In particular, the indwelling catheter may be inserted into the urethra of a human being and extend into the bladder. In this regard, the person is able to urinate through the indwelling catheter. During usage, crystals from the patient's urine along with the urine will flow through the indwelling catheter, and in some instances, the crystals may attach themselves to the inner surface of the indwelling catheter. Eventually over time, the crystals attaching themselves to the inner surface of the indwelling catheter will accumulate so as to impede the flow of urine through the indwelling catheter and eventually block the flow of urine therethrough. Upon blockage, the patient may feel great discomfort because the patient will have difficulty urinating. Accordingly, the indwelling catheter must be replaced such that the patient may continue to urinate.

The blockage of the indwelling urinary catheter may create problems with respect to time and expense. With respect to time, there may not be sufficient time to replace the indwelling catheter. The reason is that the indwelling catheter may have to be replaced every twenty four hours. In this regard there may be a shortage of medical personnel to replace the indwelling catheter because of the high demand for medical labor who are qualified to replace the indwelling catheter. Or, in other words, the patient may not be able to make an appointment with the appropriate medical personnel to replace the indwelling catheter.

With respect to expense, medical personnel who are qualified to replace the indwelling catheter must be skilled such as an LVN or an RN. These medical personnel are highly paid and as a result, the replacement of the indwelling catheter is costly. Additionally, the frequent replacement of the indwelling catheter along with high degree of skill of the medical personnel required to replace the indwelling catheter compounds and increases the cost to replace or maintain the indwelling catheter.

Moreover, blockage of indwelling catheters may create a problem with respect to discomfort inflicted on the patient each time the indwelling catheter is replaced. Simply put, indwelling catheters that have been blocked require replacement, and replacement of the indwelling catheter is uncomfortable because the indwelling catheter must be removed by sliding the indwelling catheter along the entire length of the bodily channel, and a clean replacement indwelling catheter must be inserted by sliding the replacement indwelling catheter along the entire length of the bodily channel.

Furthermore, replacement of the indwelling catheters increases the opportunity for infection. In particular, replacing the indwelling catheter provides an opportunity for the replacement catheter to be improperly installed into the bodily channel, and in this regard, the improperly installed replacement catheter creates a risk that the bodily channel will become infected which is particularly acute for females with indwelling urinary catheters.

Accordingly, there is a need for a device which will increase the amount of time between intervals at which the indwelling catheter must be replaced due to blockage. In this regard, the expense to replace blocked indwelling catheters is reduced as well as the burdens on the trained medical professionals required to replace the indwelling catheter. Additionally, increasing the amount of time between intervals at which the indwelling catheter must be replaced further reduces the frequency that the patient must endure discomfort related to removal of a blocked indwelling catheter and insertion of a clean replacement indwelling catheter. Furthermore, the risk of infection from an improperly installed indwelling catheter is reduced. Moreover, there is a need for a device to accomplish these needs discussed above that may be operated by a lay person.

### BRIEF SUMMARY OF THE INVENTION

The needs identified above have been addressed by the present invention. Generally, the present invention relates to a device which may be used to clean the indwelling catheter while the indwelling catheter is in place within a bodily channel. Accordingly, the indwelling catheter does not have to be replaced each time that it becomes blocked. In this regard, the time interval between replacements of the indwelling catheter has been increased and the discomfort due to cleaning is less than the discomfort due to replacement. Moreover, it has been found that cleaning indwelling catheters with the device of the present invention reduces the frequency of blocking, cleaning and replacing of the indwelling catheter. In other words, the device of the present invention enables the indwelling catheter to remain within the bodily channel for a longer duration of time. As such, the time and expense problems identified above have been reduced as well as the discomfort inflicted upon the patient each time the indwelling catheter is replaced and the risk of infection. Simply put, the patient experiences discomfort less frequently and the discomfort that the patient does experience through cleaning is less than the discomfort experienced through replacing the indwelling catheter.

In particular, in accordance with the present invention, there is provided a indwelling catheter cleaner comprising a head, a rod, and a handle. These three elements of the present invention may be fixedly attached to each other such that rotation of the handle may be operative to correspondingly rotate the head. Moreover, linearly pushing the handle may also be operative to linearly move the head. Simply put, the head may be manipulated by controlling the handle usually with fingers of a hand.

The head may remove debris which may be lodged within a lumen of the indwelling catheter and may be attached to an inside wall of the indwelling catheter. In particular, the head may be inserted into the indwelling catheter up to the point of the lodged debris. Thereafter, the head may be slightly pushed forward so as to hit or dislodge the debris from the inner wall. Upon removal of the cleaner from the indwelling catheter, the debris may flow therethrough. In some instances, the debris may not dislodge from the inner wall of the indwelling catheter even after the head pushes against the debris, and in this regard, the head may additionally be rotated such that cutting edges of the head may scrape off any debris that may be lodged or attached to the inner wall of the indwelling catheter.

The shape of the head may be operative to dislodge debris which may be attached to the inner wall of the indwelling catheter. In particular, the head may have an oval shape such that insertion of the head into the lumen of the indwelling catheter may simultaneously stretch the inner wall and compress the indwelling catheter at the point the head is located. The stretching of the indwelling catheter may be operative to dislodge debris from the indwelling catheter's inner wall because contact points or attachment points of crystals/debris will be broken from the stretching of the indwelling catheter tube. Moreover, the head may additionally be operative to clean not only the inside wall of the indwelling catheter at the point where the blockage occurs but also the entire linear length of the indwelling catheter. In particular, as stated above, the head may have an oval shape which when inserted into the indwelling catheter will simultaneously stretch and compress the tube. In this regard, as the head is inserted from a distal end of the indwelling catheter up to the point of blockage, the head will stretch and compress the indwelling catheter as the head is being inserted. In this regard, the head is operative to clean or dislodge debris from the inner surface of the indwelling catheter along the complete length of the indwelling catheter.

The cleaner of the present invention may be fabricated such that the head and rod is easier to insert into the indwelling catheter. In particular, the head may define a tip wherein the tip has a curved configuration such as a semi-spherical configuration. In this regard, as the head is being inserted into the indwelling catheter, the head and more particularly the tip of the head provides a smooth leading surface to reduce the coefficient of friction between the head and the inside wall of the indwelling catheter tube. Additionally, the rod outside diameter may be smaller than an inside diameter of the indwelling catheter. In this regard, this reduces the frictional forces between the rod and the inside surface of the indwelling catheter compared to a rod which may have an outside diameter which is equal to or greater than the inside diameter of the indwelling catheter. The insertion of the cleaner into the indwelling catheter may also be assisted by lubricants which may be applied to the head, and more particularly, to the tip of the head and also to the rod.

### BRIEF DESCRIPTION OF THE DRAWINGS

An illustrative and presently preferred embodiment of the invention is shown in the accompanying drawings in which:

FIG. 1 is side view of a cleaner to be inserted into an indwelling catheter wherein a head of the cleaner may extend the entire length of the indwelling catheter and has an oval shape;

FIG. 2 is a side view of the cleaner inserted into the indwelling catheter of FIG. 1;

FIG. 3 is a cut out sectional view of the head inserted into the indwelling catheter showing in greater detail debris that may be dislodged with the head of the cleaner by a pushing and rotating motion of the head;

FIG. 4 is a cross sectional view of the indwelling catheter showing a relaxed inner diameter of the indwelling catheter;

FIG. 5 is a cross sectional view of the indwelling catheter showing a head length greater than the relaxed inner diameter and a head width less than the relaxed inner diameter;

FIG. 6 is a cross sectional view of the indwelling catheter showing the head rotated 90 degrees from its position shown in FIG. 5; and

FIG. 7 is a cross sectional view of a rod of the cleaner showing that an outer diameter of the rod is smaller compared to the relaxed inner diameter of the indwelling catheter.

### DETAILED DESCRIPTION OF THE INVENTION

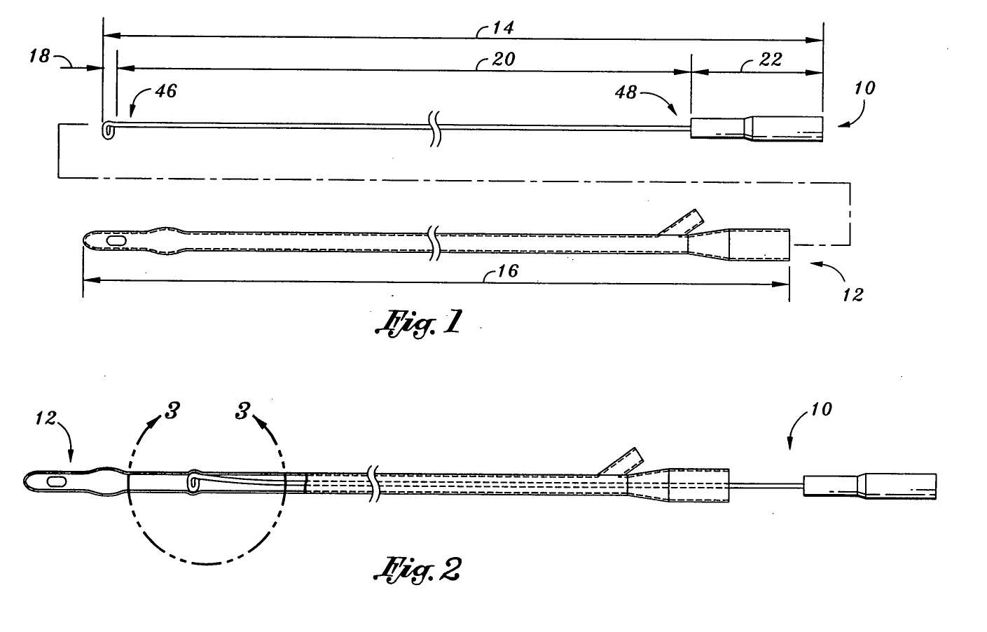

Referring now to the drawings wherein the drawings are for the purposes of illustrating the preferred embodiment of the present invention, and not for the purpose of limiting the same, FIG. 1 illustrates a side view of a cleaner 10 and a indwelling catheter 12. The cleaner, as shown in FIG. 1, illustrates a head 18, rod 20 and handle 22. And, FIG. 2 illustrates the cleaner 10 inserted into the indwelling catheter 12. In the following discussion of the present invention, an indwelling catheter 12 and its structural features shall be discussed and will be followed by a discussion of the cleaner 10 and its structural features as applied to the indwelling catheter 12. In this regard, a discussion of aspects of the present with respect to indwelling catheters are not meant to limit the aspects of the present invention discussed herein but is merely presented for illustrative purposes regarding various aspects of the present invention. For example, the cleaner 10 which is the subject matter of the present invention may be adapted to clean other types of tubes such as a flexible rubber hose.

As stated above, FIG. 2 illustrates the cleaner inserted into the indwelling catheter 12. FIG. 2 does not illustrate that the indwelling catheter 12 is inside of a bodily channel when the cleaner is inserted into the indwelling catheter 12. On this point, the present invention contemplates the use of the cleaner when the indwelling catheter 12 is outside of the bodily channel as well as while the indwelling catheter 12 is inside of the bodily channel.

The indwelling catheter 12 may typically be a long cylindrical tube defining an inner diameter 24 and an outside diameter 26 (see FIG. 3). The size of the outside diameter 26 may be sufficiently small such that the indwelling catheter 12 may be inserted into a bodily channel such as the urethra. Since the bodily channel is typically non-linear, the indwelling catheter may be fabricated from a rubber or elastic material such that a length of the indwelling catheter may be conformable to the length of the bodily channel.

The indwelling catheter 12 may define a lumen 28 (see FIG. 3) which is an inner open space of the indwelling catheter tube 28. During usage of the indwelling catheter 12 (i.e., indwelling catheter in place inside a bodily channel), frequently, debris 32 flowing therethrough blocks flow of urine therethrough. In particular, as stated above, the indwelling catheter 12 may be inserted into the urethra which provides a means to empty or drain a bladder of a person. Over a period of time, the urine that drains through indwelling catheter which also contains crystal particulate that may 32a slowly line an inside wall 34 of the indwelling catheter 12. The crystals 32a may line the inside wall 34 by attaching themselves thereto. As a result, the effective inner diameter 24 of the medical indwelling catheter 12 slowly decreases during usage such that a crystal 32a larger than the effective inner diameter 24 of the medical indwelling catheter 12 will eventually block fluidic (i.e., urine) passage therethrough.

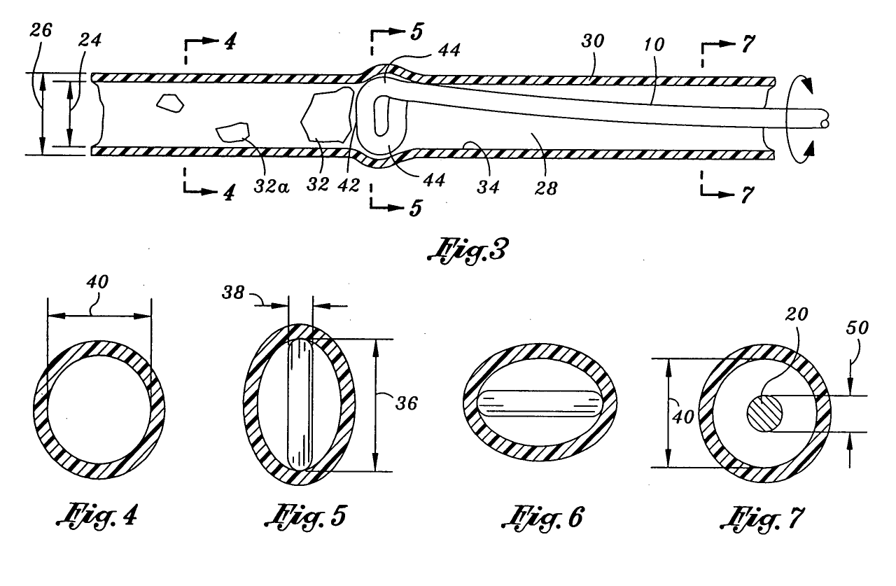

The cleaner 10 which is the subject matter of the present invention may comprise the head 18, the rod 20, and the handle 22 may be inserted into the indwelling catheter 12 to remove the debris 32 (see FIG. 3). The head 18 may define an oval cross sectional area (see FIGS. 5 and 6) wherein the cross section is in a longitudinal axis of the rod 20. The oval shape of the head 18 may define a length 36 and a width 38. The length 36 may be greater than a relaxed inside diameter 40 (see FIG. 4) of the indwelling catheter 12, and the width 38 may be less than the relaxed inside diameter 40 of the indwelling catheter 10.

The head 18 of the cleaner 10 may define a tip 42 and cutting edges 44. The tip 42 of the head 18 may have a curved configuration (see FIG. 3). The curved configuration may be a semi-spheroidal configuration, round configuration, or elliptical configuration. These listed tip configurations are merely illustrative and not exclusive of the different tip configurations which may be used in conjunction with aspects of the present invention. The curved configuration of the tip 42 may be operative to lead the cleaner 10 into the indwelling catheter 12. And, the head 18 may define the cutting edges 44. The cutting edges 44 may be formed on the head 18 below the tip 42. The cutting edges 44 may be located on the head 18 at distal opposed sides of the head 18. Preferably, the cutting edges 44 are located on the head 18 at the widest portion of the oval shape of the head 18.

The cleaner 10 may further comprise the rod 20. The rod 20 may define first and second distal ends 46, 48 (see FIG. 1). The head 18 may be attached to the first distal end 46 of the rod 20, and more particularly, the head 18 may be fixedly attached to the rod 20 such that rotation of the rod 20 along the longitudinal axis of the rod 20 may respectively rotate the head 18, as shown by FIGS. 3, 5 and 6. FIG. 3 illustrates the cleaner 10 inserted into the indwelling catheter 12 and rotateable therein. FIG. 5 illustrates the head 18 of the cleaner 10 inserted in the indwelling catheter 12, and FIG. 6 illustrates the head 18 of the cleaner 10 rotated 90 degrees.

The rod 20 may be an elongated bar and, the rod may additionally be formable yet rigid. For example, the rod may be fabricated from galvanized steel. Preferably, the rod may have a circular cross sectional area (see FIG. 7). The rod 20 may define an outside diameter 50 (see FIG. 7) which is less than the relaxed inside diameter 40 (see FIG. 7) of the tube 30 of the indwelling catheter 12.

The length 14 of the cleaner 10 may be greater than the length 16 of the indwelling catheter 12 such that any blockage that occurs within the indwelling catheter tube 30 may be removed with the cleaner 10. Since the medical indwelling catheter 12 is frequently inserted into sensitive areas of the mammal, such as the urethra, it is important that the cleaner 10, and more particularly, the head 18 does not protrude beyond the length 16 of the indwelling catheter 12. For example, if the medical indwelling catheter 12 were to be inserted into the urethra with its tip 42 extending within a bladder of the mammal, then the cleaner 10 should not extend beyond the length 16 of the medical indwelling catheter 12 such that the cleaner 10 may not puncture the bladder of the mammal.

One such method of preventing the cleaner 10 from protruding beyond the medical indwelling catheter 12 is to limit the length of the cleaner 12 (i.e., rod 20) such that the cleaner 10 may not be manipulated beyond the length of the cavity. In the alternative, markings (not shown) may be visible on the outside surface of the rod 20. The markings may correspond to a tape measure. In this regard, the cleaner 10 may be inserted into the medical indwelling catheter 12 and the insertion distance of the head 18 into the cavity may be measured by the markings that are visible from the outside surface of the rod 20. The markings may be etched into the outside surface of the rod 20. In the alternative, and more preferably, the markings may be formed under the outside surface of the rod 20. In this regard, the outside surface of the rod 20 may remain smooth such that the outside surface will not mar the inside wall of the indwelling catheter 12 during cleaning.

The head 18 and rod 20 of the cleaner 10 may be fabricated from two separate materials. For example, the head 18 may be fabricated from a hard plastic, whereas the rod 20 may be fabricated from steel. In the alternative, and more preferably, the head 18 and the rod 20 may be fabricated from a unitary material such as a single long rod. In this regard, the head 18 may be formed by slightly curling or bending the distal tip thereof to form the head 18, as shown in FIG. 3. The bend provides the curved or round configuration of the tip 42 of the head 18. Subsequently, adjacently and below the tip, the surface of the head 18 may be marred so as to form cutting edges 44. Additionally, the head 18 and/or rod 20 may be coated with a lubricant such as the lubricant commonly sold under the trademark TEFLON®.

The rod first distal end 46 may be offset from the center of the head 18, as shown in FIG. 3. In FIG. 3, the rod 20 is attached to the head 18 at one side of the oval shape of the head 18. In the alternative, the rod 20 may be attached to the head 18 at the center of the oval shape of the head 18 (not shown). In this alternative, the rod 20 may be attached to the head 18 through molding, welding or other attachment methods. Preferably, the attachment method chosen to attach the head 18 and rod 20 does not leave any burrs at its joint.

The curved configuration of the tip 42 of the head 18 and the outside diameter 50 of the rod 20 being smaller than the relaxed inside diameter 40 of the indwelling catheter 12 along with the lubricant which may be applied to the head 18 and rod 20 provide easy insertion of the cleaner 10 within the indwelling catheter 12. In particular, the curved configuration of the tip 42 of the head 18 leads the cleaner 10 within the medical indwelling catheter 12 as the cleaner 10 is being inserted therein. The lubricant on the head 18 provides additional assistance to ease the insertion of the cleaner 10 into the medical indwelling catheter 12. Lastly, the outside diameter 50 of the rod 20 being smaller compared to the relaxed inside diameter 40 of the indwelling catheter 12 provides less surface area therebetween so as to reduce the frictional forces therebetween. Moreover, the lubricant applied to the rod 20 and/or head 18 further decreases the coefficient of friction between the rod 20 and the indwelling catheter 18 to further ease the insertion of the cleaner 10 into the indwelling catheter 12.

The handle 22 may be attached to the second distal end 48 of the rod 20 (see FIG. 1). The handle 22 may have a shape such that manipulation of the cleaner 10 may be easily accomplished with the fingers of a human hand. For example, the handle 22 may be a narrow knob or a knob with indentations configured to and sized to receive the fingers of the human hand. Preferably, the handle 22 is sized and configured such that the human hand may rotate or twist the handle in the longitudinal axis of the rod 20 and traverse the handle 22 along the longitudinal axis of the rod 20. In this regard, as discussed above, the handle 22 may additionally be fixedly attached to the rod 20 and thus the rotation of the handle 22 may correspondingly rotate the rod 20 and the head 18. The handle 22 along with the rod 20 and head 18 may be fabricated from any sterilizable material such as steel or galvanized steel.

As stated above, the cleaner 10 may be operative to clear debris 32 from a lumen 28 of the medical indwelling catheter 10 while still within a bodily channel of a mammal. For example, the indwelling catheter 12 may remain within the bodily channel of the mammal during its cleaning. While the medical indwelling catheter 12 is retained within the bodily channel of the mammal, the cleaner 10 may be inserted within the lumen 28 of the indwelling catheter 12. The structure of the head 18 and rod 20 as discussed above may be operative to enable easy insertion of the cleaner 10 into the indwelling catheter 12. The cleaner 10 may continue to be inserted into the indwelling catheter 12 until the head 18 contacts debris 32 within the indwelling catheter 12 which blocks fluidic passage therethrough. An example of this situation is shown in FIG. 3. Frequently, the head 18 may be slightly pushed further so as to dislodge the debris 32 from the indwelling catheter 12. Upon removal of the cleaner 10, the debris 32 will dislodge and flow through the medical indwelling catheter 12 and the lumen 28 of the indwelling catheter 12 may be unblocked.

For debris 32 that remain lodged within the medical indwelling catheter 12 even after the slight push by the head 18, the head 18 may be rotated within the medical indwelling catheter 12 so as to rub the cutting edges 44 against the inside wall 34 of the tube. An example of the rotation is shown in FIGS. 3, 5 and 6. FIG. 5 illustrates the head 18 as it is inserted into the indwelling catheter 12, and FIG. 6 illustrates the head 18 rotating within the indwelling catheter 12. Furthermore, the head 18 may be rotated such that the head 18 may rotate at least 360 degrees. In this regard, upon removal of the cleaner 10 from the indwelling catheter 12, the debris 32 may flow through the indwelling catheter 12 and blockage of the indwelling catheter may be resolved.

The structure of the head 18 particularly, the oval shape of the head 18, may be effective to dislodge any debris 32 from the inside wall 34 of the indwelling catheter 12 along the whole length 16 which the head 18 may travel there through. The reason is that the oval shaped head 18 may be operative to simultaneously stretch the inside wall 34 as well as compress the tube 30. As stated above, the length 36 of the oval shape may be greater than the relaxed inside diameter 40 of the tube 30 (see FIGS. 4 and 5), and in this regard, upon insertion of the head 18 into the medical indwelling catheter 12, the head 18 will stretch the tube 30 along the length direction of the oval shape. Moreover, as stated above, the width 38 of the oval shape may be less than the relaxed inside diameter 40 of the tube 30 (see FIGS. 4 and 5), and in this regard, the tube 30 may be compressed along the width direction of the oval shape of the head 18. Simply put, the insertion of the head 18 may be effective in dislodging debris 32 which may be physically attached to the inside wall 34 of the tube 30.

This description of the various embodiments of the present invention is presented to illustrate the preferred embodiments of the present invention, and other inventive concepts may be otherwise variously embodied and employed. The appended claims are intended to be construed to include such variations except insofar as remitted by the prior art.

1. A cleaner for removing debris within a lumen of a tube, the cleaner comprising:
a. a head having an oval shape for simultaneously stretching and compressing the tube;
b. a rod defining first and second distal ends, the first distal end being attached to the head; and
c. a handle attached to the second distal end of the rod.

2. The cleaner of claim 1 wherein the oval shape is in a cross section of the head along a longitudinal axis of the rod.

3. The cleaner of claim 2 wherein the oval shaped head defines a length that is greater than a relaxed inside diameter of the tube.

4. The cleaner of claim 1 wherein an outside diameter of the rod is less than a relaxed inside diameter of the tube.

5. The cleaner of claim 1 wherein a tip of the head is curved.

6. The cleaner of claim 5 wherein the tip of the head is round.

7. The cleaner of claim 1 wherein cutting edges are formed on opposed sides of the oval shape of the head.

8. The cleaner of claim 1 wherein the rod and head are coated with a lubricant.

9. The cleaner of claim 1 wherein the rod is semi flexible.

10. The cleaner of claim 1 wherein the handle, rod and head are in fixed relationship with each other such that rotating of the handle rotates the head to remove debris from an inner wall of the tube.

11. The cleaner of claim 1 wherein the tube is a catheter.

12. The cleaner of claim 1 wherein the rod has visible measurement markings.

13. A method of removing debris from within a tube, the method comprising the steps of:
a. providing a cleaner having an oval shaped head and a rod attached to the oval shaped head;
b. inserting the oval shaped head within the tube until the head contacts the debris; and
c. pushing the debris with the oval shaped head to loosen the debris from within the tube.

14. The method of claim 13 further comprising the step of rotating the oval shaped head to simultaneously stretch a sidewall of the tube, compress the tube and rub the sidewall of the tube with cutting edges of the head.

15. The method of claim 14 wherein the rotating step is accomplished after the pushing step.
